(19) United States
(12) Patent Application Publication
(10) Pub. No.: US 2024/0189386 A1
(43) Pub. Date: Jun. 13, 2024

(54) COMPOSITION FOR ANTI-FATIGUE COMPRISING GREEN TEA PEPTIDE COMPOSITION

(71) Applicant: AMOREPACIFIC CORPORATION, Seoul (KR)

(72) Inventors: Hyun Woo JEONG, Yongin-si (KR); Jinoh CHUNG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/515,425

(22) Filed: Nov. 21, 2023

(30) Foreign Application Priority Data
Dec. 9, 2022 (KR) 10-2022-0171257

(51) Int. Cl. A61K 38/16; A61K 36/82; A61K 38/08; A61K 38/10; A61P 19/00

(52) U.S. Cl. CPC A61K 38/168; A61K 36/82; A61K 38/08; A61K 38/10; A61P 19/00; A61K 2236/19; A61K 2236/333; A61K 2236/39

(57) ABSTRACT

The present disclosure relates to a composition for anti-fatigue comprising a green tea peptide composition. Specifically, the green tea peptide composition according to the present disclosure exhibits anti-fatigue efficacy through excellent effects of inhibiting production of fatigue substances within a muscle cell, increasing ATP production within the muscle cell, and promoting fat oxidation within the muscle cell, and can be applied to various health functional food compositions and pharmaceutical compositions.

### CROSS REFERENCE TO RELATED APPLICATION

The present application claims the priority of Korean Patent Application No. 10-2022-0171257, filed on Dec. 9, 2022, the entire contents of which is incorporated herein for all purposes by this reference.

The “Sequence Listing” submitted electronically concurrently herewith pursuant 37 C.F.R. § 1.821 in computer readable form (CRF) as file name 3549-134Seq.XML is incorporated herein by reference. The electronic copy of the Sequence Listing was created on Oct. 19, 2023, and the size on disk is 8,192 bytes.

### BACKGROUND OF THE INVENTION

### Technical Field

The present disclosure relates to a composition for anti-fatigue comprising a green tea peptide composition.

### Background

A tea tree is one of 82 species classified in the genus Camellia, and is currently cultivated in over fifty (50) countries, mainly in Asia, including Africa, South America, and Oceania. Types of the tea are largely classified into non-fermented tea, semi-fermented tea, fermented green tea, and post-fermented tea depending on the processing method of the tea tree leaves. Among them, the non-fermented tea is made by inactivating polyphenol oxidase contained in the tea tree through heat treatment. Compared to other teas, the non-fermented tea contains more polyphenols such as flavonol, flavanone, and flavonoid, which show strong antioxidant power and account for about 30% based on the dry weight of the tea.

As a pharmacological mechanism of various components contained in the green tea have gradually been revealed, its value has been recognized by the general public. In particular, polyphenol as the main component of the green tea is attracting great attention as it has been proven to have efficacy such as antioxidant action, anti-cancer action, blood cholesterol-lowering action, anti-aging action, heavy metal detoxification action, cavity prevention, and bad breath removal action.

Since peptide is stable against oxidation among the substances contained in plants and has a simple structure, it is expected to have high efficacy to a skin. The peptide in the plants acts as a signaling substance, and is known to be particularly involved in growth and differentiation of the plants, and response to external stimuli.

Meanwhile, fatigue is divided into mental and physical fatigue. In general, the physical fatigue is defined as a condition in which the force required for muscle contraction activity cannot be sufficiently exerted or maintained, a condition in which physical or mental ability is reduced to the extent that it cannot be recovered by rest due to overwork or energy depletion, and a condition in which work efficiency or exercise performance is decreased, thereby causing symptoms such as languorousness, tiredness, exhaustion, drowsiness, difficulty concentrating, headache, and muscle pain.

Cause of the physical fatigue is divided into deficiency or inability to use energy source stored in the body, accumulation of fatigue substances due to metabolism, and loss of homeostasis in the body. However, the physical fatigue is not caused by a single factor, but is a characteristic caused by the synergistic action of many factors. Continuous exercise causes the fatigue, and such a fatigue can be inhibited by the intake of nutritional supplements, thereby suppressing fatigue relevance factors. During exercise, many tissues help remove a lactic acid from the blood, but the exercise of high intensity produces the lactic acid at a higher rate than the tissue can eliminates it, which can cause the fatigue. The lactic acid accumulated in the blood is the fatigue relevance factor.

Recovery from such physical fatigue requires sufficient supply of energy sources, rest, and inhibition and removal of the fatigue substances in the body. However, in reality, sufficient intake of the nutrition and the rest are not achieved in the busy modern society. Therefore, there is a need to develop functional substances that can recover or suppress the fatigue.

### SUMMARY OF THE INVENTION

The purpose of the present disclosure is to provide a composition that exhibits excellent anti-fatigue efficacy, comprising as an active ingredient a green tea peptide having a novel amino acid sequence isolated and purified through culture of a green tea protein and specific vegetable lactic acid bacterium.

In order to achieve the above purpose, an embodiment of the present invention provides a composition for anti-fatigue comprising a green tea peptide composition as an active ingredient, wherein the green tea peptide composition comprises one or more green tea peptides containing an amino acid sequence selected from the group consisting of SEQ ID NO: 1 to SEQ ID NO: 7.

The green tea peptide composition according to the present disclosure exhibits anti-fatigue efficacy through excellent effects of inhibiting production of fatigue substances within a muscle cell, increasing ATP production within the muscle cell, and promoting fat oxidation within the muscle cell, and can be applied to various health functional food compositions and pharmaceutical compositions.

### BRIEF DESCRIPTION OF THE DRAWINGS

FIG. 1 schematically shows a process of preparing a green tea peptide according to an embodiment of the present invention.

FIGS. 2A to 2C show the results confirming the effects of inhibiting production of fatigue substances in a muscle cell at various concentrations of a green tea peptide (GTP) composition according to Experimental Example 1 (***P<0.001 vs. CoCl₂, **P<0.01 vs. CoCl₂, *P<0.05 vs. CoCl₂).

FIGS. 3A and 3B show the results of confirming the effects of inhibiting LDHA activity and accumulation of a lactic acid in a muscle cell at various concentrations of a green tea peptide (GTP) composition according to Experimental Example 1 (***P<0.001 vs. CoCl₂, **P<0.01 vs. CoCl₂, *P<0.05 vs. CoCl₂).

FIG. 4 shows the results of confirming the effect of increasing ATP production in a muscle cell at various concentrations of a green tea peptide (GTP) composition according to Experimental Example 2 (***P<0.001 vs. (−), **P<0.01 vs. (−)).

FIG. 5 shows the results of confirming the effect of increasing mitochondria in a muscle cell at various concentrations of a green tea peptide (GTP) composition according to Experimental Example 2 (***P<0.001 vs. (−), *P<0.05 vs. (−)).

FIGS. 6A to 6D show the results of confirming the effect of increasing expression of genes involved in the mitochondrial electron transport system within a muscle cell at various concentrations of a green tea peptide (GTP) composition according to Experimental Example 2 (***P<0.001 vs. (−), **P<0.01 vs. (−)).

FIGS. 7A to 7D show the results of confirming the effects of promoting fat oxidation in a muscle cell at various concentrations of a green tea peptide (GTP) composition according to Experimental Example 3 (***P<0.001 vs. (−), **P<0.01 vs. (−), *P<0.05 vs. (−)).

FIG. 8 shows the results of comparing the effect of a green tea peptide on inhibiting accumulation of a lactic acid in a muscle cell by the processing method according to Experimental Example 4 (***P<0.001 vs. CoCl₂, **P<0.01 vs. CoCl₂, *P<0.05 vs. CoCl₂).

FIG. 9 shows the results of comparing the similarity between Lacticaseibacillus paracasei and Lactiplantibacillus plantarum.

FIGS. 10A and 10B show the results of molecular weight analysis of a green tea protein decomposition product by Lacticaseibacillus paracasei and Lactiplantibacillus plantarum, respectively.

FIG. 11 shows the results of comparing the effect of inhibiting a lactic acid accumulation at various molecular weight fractions of a green tea peptide (GTP) composition according to Experimental Example 5 (***P<0.001 vs. CoCl₂, **P<0.01 vs. CoCl₂, *P<0.05 vs. CoCl₂).

### DETAILED DESCRIPTION OF THE INVENTION

Hereinafter, the present invention will be described in detail.

In this specification, a “green tea (Camellia Sinensis)” is an evergreen broad-leaved shrub belonging to the Camellia family, and a tea from which the leaves are dried is used in various fields. In particular, it is known to exhibit antioxidant action, anti-cancer action, and blood lipid-reducing action and blood circulation-promoting action in the cardiovascular system. The green tea may include at least one selected from the group consisting of a leaf, a flower, a stem, a fruit, a root, a stem, and a heartwood of the root, and may preferably be the leaf.

In this specification, an “active ingredient” refers to an ingredient that exhibits the desired activity alone or can exhibit the activity in combination with a carrier that is not active on its own.

As used herein, an “anti-fatigue” means preventing or improving fatigue, or alleviating, recovering, or eliminating physical fatigue by preventing or delaying accumulation of the physical fatigue. Examples of the physical fatigue may include a condition in which the force required for muscle contraction activity cannot be sufficiently exerted or maintained, a condition in which physical or mental ability is reduced to the extent that it cannot be recovered by rest due to overwork or energy depletion, chronic fatigue syndrome (CFS), a condition in which clinically unexplained fatigue occurs continuously or repeatedly for more than 6 months without a specific cause disease and seriously affects daily life, or a condition in which work efficiency or exercise performance is decreased, thereby causing symptoms such as decrease in cognitive ability, sleep disorder, languorousness, tiredness, exhaustion, drowsiness, difficulty concentrating, headache, myofascial pain syndrome, and tenderness in a specific area of a muscle (muscle pain).

Specifically, the fatigue is caused by a lack of phosphocreatinine in the muscle, accumulation of a proton in the muscle (acidemia), a lack of glucagon in the muscle, decrease in a concentration of glucose in the blood, and increase in a ratio of a specific amino acid in a plasma, etc., which results in affecting peripheral and central nervous fatigue. The central nervous fatigue means that activity of a skeletal muscle decreases, whereas the peripheral nervous fatigue refers to change in a neuromuscular junction.

In an aspect, the present invention relates to a composition for anti-fatigue comprising a green tea peptide composition as an active ingredient, wherein the green tea peptide composition comprises one or more green tea peptides containing an amino acid sequence selected from the group consisting of SEQ ID NO: 1 to SEQ ID NO: 7.

In other aspect, the present invention is directed to a method for preventing, improving, alleviating, recovering, or eliminating fatigue, comprising administering to a subject in need an effective amount of a green tea peptide composition comprising one or more green tea peptides containing an amino acid sequence selected from the group consisting of SEQ ID NO: 1 to SEQ ID NO: 7.

In another aspect, the present invention is concerned to a use of a green tea peptide composition for preparing a composition for anti-fatigue, wherein the green tea peptide composition comprises one or more green tea peptides containing an amino acid sequence selected from the group consisting of SEQ ID NO: 1 to SEQ ID NO: 7.

In an embodiment, the green tea peptide composition may comprise one or more green tea peptides containing an amino acid sequence selected from the group consisting of SEQ ID NO: 1 to SEQ ID NO: 7. Specifically, the green tea peptides may include an amino acid sequence selected from the group consisting of AYKRRKGKFA (SEQ ID NO: 1), FFFFFFFFFFFFFFFYL (SEQ ID NO: 2), ISKIWNSEVPETEVKNEAESP (SEQ ID NO: 3), PFFCEKMMETN (SEQ ID NO: 4), RFLHERMAYYH (SEQ ID NO: 5), RNLNRLQRLLSMKQEYSPRNHLGSRWREY (SEQ ID NO: 6), and TTSSRKKEKPRRFWNNHEEVFLITTK (SEQ ID NO: 7).

In an embodiment, the one or more green tea peptides account for 60% (w/w) or more, 65% (w/w) or more, 70% (w/w) or more, 75% (w/w) or more, 80% or more, 85% (w/w) or more, 90% (w/w) or more, or 95% (w/w) or more of the green tea peptide composition.

In an embodiment, the green tea peptide composition may be obtained by fermenting a green tea protein with vegetable lactic acid bacterium.

In an embodiment, the fermentation may be carried out under a condition of pH 5 to 8. Specifically, the fermentation may be performed at pH 5 or higher, pH 5.2 or higher, pH 5.4 or higher, pH 5.6 or higher, pH 5.8 or higher, pH 6 or higher, pH 6.2 or higher, pH 6.4 or higher, pH 6.6 or higher, pH 6.8 or higher, pH 7 or higher, pH 7.2 or higher, pH 7.4 or higher, pH 7.6 or higher, or pH 7.8 or higher, and the fermentation may also be performed at pH 8 or lower, pH 7.8 or lower, pH 7.6 or lower, pH 7.4 or lower, pH 7.2 or lower, pH 7 or lower, pH 6.8 or lower, pH 6.6 or lower, pH 6.4 or lower, pH 6.2 or lower, pH 6 or lower, pH 5.8 or lower, pH 5.6 or lower, pH 5.4 or lower, or pH 5.2 or lower. Preferably, the fermentation may be carried out at pH 6.8.

In an embodiment, the fermentation may be carried out at 25° C. to 45° C. Specifically, the fermentation may be carried out at 25° C. or higher, 27° C. or higher, 29° C. or higher, 31° C. or higher, 33° C. or higher, 35° ° C. or higher, 37° ° C. or higher, 39° C. or higher, 41° C. or higher, or 43° C. or higher, Additionally, the fermentation may be carried out at 45° C. or lower, 43° C. or lower, 41ºC or lower, 39ºC or lower, 37° C. or lower, 35° ° C. or lower, 33° C. or lower, 31° C. or lower, 29° C. or lower, or 27° C. or lower. Preferably, the fermentation may be carried out at 37° C.

In an embodiment, the fermentation may performed for 24 to 72 hours. Specifically, the fermentation may be performed for 24 hours or more, 26 hours or more, 28 hours or more, 30 hours or more, 32 hours or more, 34 hours or more, 36 hours or more, 38 hours or more, 40 hours or more, 42 hours or more, 44 hours or more, 46 hours or more, 48 hours or more, 50 hours or more, 52 hours or more, 54 hours or more, 56 hours or more, 58 hours or more, 60 hours or more, 62 hours or more, 64 hours or more, 66 hours or more, 68 hours or more, or 70 hours or more. In addition, the fermentation may be performed for 72 hours or less, 70 hours or less, 68 hours or less, 66 hours or less, 64 hours or less, 62 hours or less, 60 hours or less, 58 hours or less, 56 hours or less, 54 hours or less, 52 hours or less, 50 hours or less, 48 hours or less, 46 hours or less, 44 hours or less, 42 hours or less, 40 hours or less, 38 hours or less, 36 hours or less, 34 hours or less, 32 hours or less, 30 hours or less, 28 hours or less, or 26 hours or less. Preferably, the fermentation can be performed for 48 hours.

In an embodiment, the vegetable lactic acid bacterium may be Lactiplantibacillus plantarum. More specifically, the vegetable lactic acid bacterium may be Lactiplantibacillus plantarum APsulloc 331261 (Korean Culture Center of Microorganisms, accession number KCCM11179P, accession date 20110328).

In an embodiment, the green tea protein may be obtained from a residue of a primary extract of the green tea extracted with anhydrous or hydrous C1-C6 lower alcohol.

In an embodiment, an alcohol concentration in the hydrous C1-C6 lower alcohol may be 20 to 80% (v/v). Specifically, the alcohol concentration in the hydrous C1-C6 lower alcohol may be 20% (v/v) or more, 22% (v/v) or more, 24% (v/v) or more, 26% (v/v) or more, 28% (v/v) or more, 30% (v/v) or more, 32% (v/v) or more, 34% (v/v) or more, 36% (v/v) or more, 38% (v)/v) or more, 40% (v/v) or more, 42% (v/v) or more, 44% (v/v) or more, 46% (v/v) or more, 48% (v/v) or more, 50% (v/v) or more, 52% (v/v) or more, 54% (v/v) or more, 56% (v/v) or more, 58% (v/v) or more, 60% (v/v) or more, 62% (v/v) or more, 64% (v/v) or more, 66% (v/v) or more, 68% (v/v) or more, 70% (v/v) or more, 72% (v/v) or more, 74% (v/v) or more, 76% (v/v) or more, or 78% (v/v) or more, and may also be 80% (v/v) or less, 78%. (v/v) or less, 76% (v/v) or less, 74% (v/v) or less, 72% (v/v) or less, 70% (v/v) or less, 68% (v/v) or less, 66% (v/v) or less, 64% (v/v) or less, 62% (v/v) or less, 60% (v/v) or less, 58% (v/v) or less, 56% (v/v) or less, 54% (v/v) or less, 52% (v/v) or less, 50% (v/v) or less, 48% (v/v) or less, 46% (v/v) or less, 44% (v/v) or less, 42% (v/v) or less, 40% (v/v) or less, 38% (v/v) or less, 36% (v/v) or less, 34% (v)/v) or less, 32% (v/v) or less, 30% (v/v) or less, 28% (v/v) or less, 26% (v/v) or less, 24% (v/v) or less, or 22% (v/v) or less.

In an embodiment, the hydrous C1-C6 lower alcohol may be 20 to 80% (v/v) ethanol aqueous solution. Specifically, the hydrous C1-C6 lower alcohol may be 20% (v/v) ethanol aqueous solution, 25% (v/v) ethanol aqueous solution, 30% (v/v) ethanol aqueous solution, 35% (v/v) ethanol aqueous solution, 40% (v/v) ethanol aqueous solution, 41% (v/v) ethanol aqueous solution, 42% (v/v) ethanol aqueous solution, 43% (v/v) ethanol aqueous solution, 44% (v/v) ethanol aqueous solution, 45% (v/v) ethanol aqueous solution, 46% (v/v) ethanol aqueous solution, 47% (v/v) ethanol aqueous solution, 48% (v/v) ethanol aqueous solution, 49% (v/v) ethanol aqueous solution, 50% (v/v) ethanol aqueous solution, 51% (v/v) ethanol aqueous solution, 52% (v/v) ethanol aqueous solution, 53% (v/v) ethanol aqueous solution, 54% (v/v) ethanol aqueous solution, 55% (v/v) ethanol aqueous solution, 56% (v/v) ethanol aqueous solution, 57% (v/v) ethanol aqueous solution, 58% (v/v) ethanol aqueous solution, 59% (v/v) ethanol aqueous solution, 60% (v/v) ethanol aqueous solution, 65% (v/v) ethanol aqueous solution, 70% (v/v) ethanol aqueous solution, 75% (v/v) ethanol aqueous solution, or 80% (v/v) ethanol aqueous solution.

In an embodiment, the green tea protein may be obtained from a residue of a secondary extract obtained by extracting the residue of the primary extract with thermal water.

In an embodiment, the green tea protein may be obtained from the residue of the secondary extract through alkali extraction, filtration, and acid precipitation.

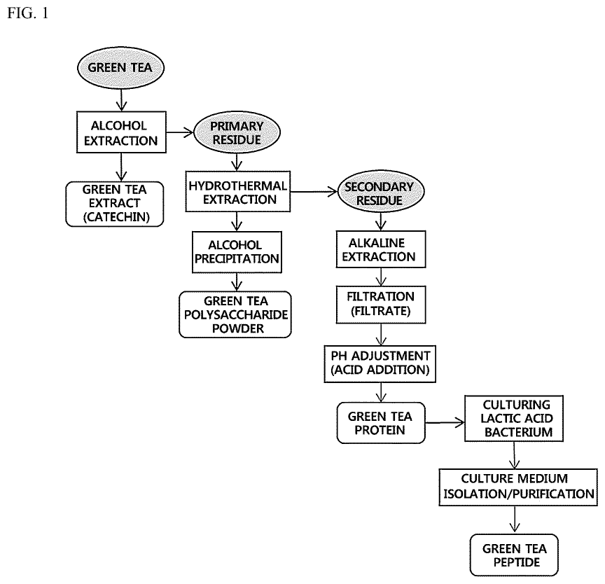

As shown in FIG. 1, a green tea peptide composition according to an embodiment of the present invention may be prepared by first extracting a green tea with an alcohol followed by second extracting a residue remaining after the first extraction with thermal water, and then cultivating a lactic acid bacterium on the green tea protein obtained through alkaline extraction, filtration, and acid precipitation of a residue remaining after the secondary extraction.

In an embodiment, the green tea peptide composition can inhibit production of a fatigue substance in a muscle cell.

In an embodiment, the fatigue substance may be a lactic acid.

In an embodiment, the green tea peptide composition may reduce expression of any one or more of LDHA (lactate dehydrogenase A) and PDK (pyruvate dehydrogenase kinase).

In an embodiment, the green tea peptide composition may increase expression of PDH (pyruvate dehydrogenase).

Specifically, the green tea peptide composition can inhibit accumulation of the lactic acid in the muscle cell by reducing expression of any one or more of LDHA and PDK. Additionally, the green tea peptide composition can inhibit accumulation of the lactic acid in the muscle cell by increasing expression of PDH.

In an embodiment, the green tea peptide composition can promote fat oxidation within the muscle cell.

In an embodiment, the green tea peptide composition may increase expression of any one or more of Acyl-CoA oxidase (ACO), Carnitine-palmitoyl transferase (CPT), Medium-chain acyl-CoA dehydrogenase (mCAD), and Peroxisome proliferator-activated receptor alpha (PPARα).

Specifically, the green tea peptide composition can promote fat oxidation in the muscle cell by increasing expression of any one or more of ACO, CPT, mCAD, and PPARα.

In an embodiment, the green tea peptide composition can promote mitochondrial biosynthesis in the muscle cell.

In an embodiment, the green tea peptide composition can increase expression of any one or more of Mitochondrial transcription factor A (TFAM), NADH dehydrogenase [ubiquinone] 1 alpha subcomplex subunit 9 (NDUFA9), Cytochrome c oxidase subunit 4 (COX4), and Adenosine triphosphate synthase, mitochondrial F1 complex, subunit alpha (ATP5a).

Specifically, the green tea peptide composition can promote mitochondrial biogenesis in the muscle cell by increasing expression of any one or more of TFAM, NDUFA9, COX4, ATP5a, and UCP2.

In an embodiment, the green tea peptide composition can promote ATP biosynthesis in the muscle cell.

Specifically, the green tea peptide composition can promote ATP synthesis by increasing mitochondria in the muscle cell.

In an embodiment, the green tea peptide composition may be comprised in an amount of 1 to 50 wt % based on the total weight of the anti-fatigue composition. Specifically, the green tea peptide composition may be present in an amount of 1 wt % or more, 3 wt % or more, 5 wt % or more, 7 wt % or more, 10 wt % or more, 12 wt % or more, 14 wt % or more, 16 wt % or more, 18 wt % or more, 20 wt % or more, 22 wt % or more, 24 wt % or more, 26 wt % or more, 28 wt % or more, 30 wt % or more, 32 wt % or more, 34 wt % or more, 36 wt % or more, 38 wt % or more, 40 wt % or more, 42 wt % or more, 44 wt % or more, 46 wt % or more, or 48 wt % or more, based on the total weight of the anti-fatigue composition. Further, the green tea peptide composition may be present in an amount of 50 wt % or less, 48 wt % or less, 46 wt % or less, 44 wt % or less, 42 wt % or less, 40 wt % or less, 38 wt % or less, 36 wt % or less, 34 wt % or less, 32 wt % or less, 30 wt % or less, 28 wt % or less, 26 wt % or less, 24 wt % or less, 22 wt % or less, 20 wt % or less, 18 wt % or less, 16 wt % or less, 14 wt % or less, 12 wt % or less, 10 wt % or less, 8 wt % or less, 7 wt % or less, 5 wt % or less, or 3 wt % or less, based on the total weight of the anti-fatigue composition.

In an embodiment, the green tea peptide composition may be administered in an amount of 1 to 400 mg/kg/day. Specifically, the green tea peptide composition may be administered in an amount of 1 mg/kg/day or more, 5 mg/kg/day or more, 10 mg/kg/day or more, 20 mg/kg/day or more, 30 mg/kg/day or more, 40 mg/kg/day or more, 50 mg/kg/day or more, 60 mg/kg/day or more, 70 mg/kg/day or more, 80 mg/kg/day or more, 90 mg/kg/day or more, 100 mg/kg/day or more, 150 mg/kg/day or more, 200 mg/kg/day or more, 250 mg/kg/day or more, 300 mg/kg/day or more, or 350 mg/kg/day or more. Further, the green tea peptide composition may be administered in an amount of 400 mg/kg/day or less, 350 mg/kg/day or less, 300 mg/kg/day or less, 250 mg/kg/day or less, 200 mg/kg/day or less, 150 mg/kg/day or less, 100 mg/kg/day or less, 90 mg/kg/day or less, 80 mg/kg/day or less, 70 mg/kg/day or less, 60 mg/kg/day or less, 50 mg/kg/day or less, 40 mg/kg/day or less, 30 mg/kg/day or less, 20 mg/kg/day or less, 10 mg/kg/day or less, or 5 mg/kg/day or less.

In an embodiment, the anti-fatigue composition may be a pharmaceutical or food composition. More specifically, the composition may be a pharmaceutical composition for preventing, improving, alleviating, recovering, or eliminating fatigue, or a health functional food composition for anti-fatigue.

The food composition may be formulated, for example, into a tablet, a granule, a pill, a powder, liquid such as a drink, a caramel, a gel, a bar, a tea bag, etc., but is not limited thereto. In addition to the active ingredients, the food composition within each formulation may be appropriately selected from and mixed with the ingredients commonly used in the field without difficulty by a person skilled in the art according to the intended formulation or the purpose of use, and the composition may exert a synergistic effect if applied simultaneously with other raw materials.

The composition may be administered in several ways, such as simple ingestion, drinking, injection, spray administration, or squeeze administration.

The food composition according to an aspect of the present invention may, for example, be healthy functional food products including various food products such as a chewing gum, a caramel product, a candy, an ice cream, and a confectionery, beverage products such as a soft drink, a mineral water, and an alcoholic beverage, and health products such as a vitamin and a mineral.

The food composition according to an aspect of the present invention may contain a food additive in addition to the active ingredients. The food additive can generally be understood as a substance that is added to, mixed with, or infiltrated into a food when preparing, processing, or preserving the food. Since the food composition is ingested daily and for a long period of time with the food, its safety must be guaranteed. In the Food Additive Code of Law of each country that governs the preparation and distribution of the food (the Food Sanitation Act in Korea), the food additive with guaranteed safety is limited in terms of its ingredient or function. In the Korean Food Additive Code (the “Standard and Specification of Food Additive” notified by the Ministry of Food and Drug Safety), the food additive is classified into a chemical synthesis, a natural additive, and a mixed preparation in terms of its ingredient. Such a food additive is classified into a sweetener, a flavoring agent, a preservative, an emulsifier, an acidulant, a thickener, etc., in terms of its function.

The sweetener is used to impart appropriate sweetness to a food, and both natural and synthetic sweeteners may be used in the food composition according to an aspect of the present invention. The natural sweetener is preferably used, and may include sugar sweeteners such as a corn syrup solid, a honey, sucrose, fructose, lactose, and maltose.

The flavoring agent is used to enhance taste or aroma, and both natural and synthetic flavoring agents may be used. The natural flavoring agent is preferably used. In the case of using the natural flavoring agent, it may serve the purpose of strengthening nutrition in addition to the flavor. The natural flavoring agent may be obtained from an apple, a lemon, a tangerine, a grape, a strawberry, a peach, etc., or may be obtained from a green tea leaf, a solomon's seal leaf, a bamboo leaf, a cinnamon, a chrysanthemum leaf, a jasmine, etc. The natural flavoring agent may also be obtained from a ginseng (a red ginseng), a bamboo shoot, an aloe vera, and a ginkgo nut. The natural flavoring agent may be a liquid concentrate or a solid extract. The synthetic flavoring agent may be used, if necessary, and may include ester, alcohol, aldehyde, terpene, etc.

The preservative may include calcium sorbate, sodium sorbate, potassium sorbate, calcium benzoate, sodium benzoate, potassium benzoate, EDTA (ethylenediaminetetraacetic acid), etc. Further, the emulsifier may include an acacia gum, a carboxymethyl cellulose, a xanthan gum, and pectin. etc., and the acidulant may include citric acid, malic acid, fumaric acid, adipic acid, phosphoric acid, gluconic acid, tartaric acid, ascorbic acid, acetic acid, phosphoric acid, etc. In addition to the purpose of enhancing taste, the acidulants may be added to ensure that the food composition has an appropriate acidity for the purpose of suppressing proliferation of the microorganisms. The thickener may include a suspending agent, a settling agent, a gel forming agent, a bulking agent, etc.

In addition to the food additive described above, the food composition according to an aspect of the present invention may contain a biologically active substance or a mineral which has been known in the art and whose safety has been guaranteed as the food additive for the purpose of supplementing and reinforcing functionality and nutrition.

Such a biologically active substance may include catechin contained in the green tea, vitamin such as vitamin B1, vitamin C, vitamin E, and vitamin B12, tocopherol, dibenzoylthiamine, etc. The mineral may include a calcium preparation such as calcium citrate, a magnesium preparation such as magnesium stearate, an iron preparation such as iron citrate, chromium chloride, potassium iodide, selenium, germanium, vanadium, zinc, etc.

The food composition according to an aspect of the present invention may contain the above-described food additive in an appropriate amount to achieve the purpose of the addition depending on the types of the product.

The other food additives that may be contained in the food composition according to an aspect of the present invention can be referred to each country's food code or food additive code.

A pharmaceutical composition according to an aspect of the present invention comprises a pharmaceutically acceptable carrier thereof in addition to the active ingredients, and can be prepared into an oral formulation or a parenteral formulation depending on the route of administration by a conventional method known in the art. Herein, the route of administration may be any suitable route, including topical route, oral route, intravenous route, intramuscular route, and direct absorption through a mucosal tissue, and the administration may include combination of the two or more routes. An example of combination of the two or more routes is that two or more dosage forms of drugs are combined according to the route of administration, for example, one drug is administered firstly through the intravenous route and the other drug is administered secondarily through the topical route.

The pharmaceutically acceptable carrier is well known in the art depending on the route of administration or dosage form, and specifically, reference can be made to the pharmacopoeia of each country, including the “Korean Pharmacopoeia”.

When the pharmaceutical composition according to an aspect of the present invention is prepared as the oral formulation, the oral formulation may be made of a powder, a granule, a tablet, a pill, a sugar-coated tablet, a capsule, a solution, a gel, a syrup, a suspension, a wafer, etc., together with a suitable carrier according to the method known in the art. In this case, an example of the suitable carrier includes saccharides such as lactose, glucose, sucrose, dextrose, sorbitol, mannitol, and xylitol, starches such as a corn starch, a potato starch, and a wheat starch, celluloses such as cellulose, methylcellulose, ethylcellulose, sodium carboxymethylcellulose, and hydroxypropylmethylcellulose, polyvinyl pyrrolidone, water, methylhydroxybenzoate, propylhydroxybenzoate, magnesium stearate, a mineral oil, a malt, a gelatin, a talc, polyol, a vegetable oil, ethanol, glycerol, etc. The formulation may contain a binder, a lubricant, a disintegrant, a colorant, a diluent, etc., if necessary. The suitable binder includes a starch, magnesium aluminum silicate, starch ferrist, gelatin, methylcellulose, sodium carboxymethylcellulose, polyvinylpyrrolidone, glucose, a corn sweetener, sodium alginate, polyethylene glycol, a wax, etc., and the lubricant includes sodium oleate, sodium stearate, magnesium stearate, sodium benzoate, sodium acetate, sodium chloride, silica, talcum, stearic acid, magnesium and calcium salts thereof, and polyethylene glycol, etc. The disintegrant includes methyl cellulose, an agar, a bentonite, a xanthan gum, alginic acid, or a sodium salt thereof, etc. Further, the diluent includes lactose, dextrose, sucrose, mannitol, sorbitol, cellulose, glycine, etc.

When the pharmaceutical composition according to an aspect of the present invention is prepared as the parenteral formulation, the parenteral formulation may be made in the form of an injection, a transdermal administration, a nasal inhalant, and a suppository, together with a suitable carrier according to the method known in the art. When formulated as the injection, the suitable carrier may include an aqueous isotonic solution or a suspension. Specifically, the isotonic solution may include phosphate buffered saline (PBS) containing triethanolamine, sterile water for injection, or 5% dextrose. The transdermal administration may be formulated in the form of an ointment, a cream, a lotion, a gel, an external solution, a pasta preparation, a liniment preparation, and an airol preparation. The nasal inhalant may be formulated in the form of an aerosol spray using a suitable propellant such as dichlorofluoromethane, trichlorofluoromethane, dichlorotetrafluoroethane, and carbon dioxide. When formulated as the suppository, the carrier may include Witepsol, Tween 61, polyethylene glycol, a cocoa fat, a laurel fat, polyoxyethylene sorbitan fatty acid ester, polyoxyethylene stearate, sorbitan fatty acid ester, etc.

An applied amount or dosage of the pharmaceutical composition according to an aspect of the present invention will vary depending on the age, gender, weight, pathological condition and severity of a subject, the route of administration, or judgment of a prescriber. Determining a dosage of the active ingredients based on these factors is within the level of those skilled in the art.

In addition to the active ingredients, the anti-fatigue composition according to an aspect of the present invention may further comprise any compound or natural extract whose safety has already been proven in the art and known to have the corresponding activity so as to increase and reinforce the efficacy of anti-fatigue or to improve the convenience of taking or ingesting through addition of the similar activity such as blood pressure-regulating activity. Such a compound or extract includes compounds or extracts listed in the compendium documents such as the Pharmacopoeia of each country (the “Korean Pharmacopoeia” in Korea) and the Code for Health Functional Foods in each country (the “Standards and Specification for Health Functional Foods” notified by the Ministry of Food and Drug Safety in Korea), compounds or extracts that have received product approval in accordance with the laws of each country governing the preparation and sale of pharmaceuticals (the “Pharmaceutical Affairs Act” in Korea), or compounds or extracts whose functionality has been recognized in accordance with the laws of each country governing the preparation and sale of health functional foods (the “Health Functional Foods Act” in Korea).

For example, according to the “Health Functional Foods Act” in Korea, the above compound or extract would correspond to complexes such as Garcinia cambogia bark extract, conjugated linolenic acid (free fatty acid), conjugated linolenic acid (triglyceride), green tea extract, chitosan, Lactobacillus gasseri BNR17, L-carnitine tartrate, green mate extract, green coffee bean extract, perilla leaf extract, and soybean embryo extract, which have been recognized for their functionality as “reduction in the body fat”; complex extracts (xanthigen) such as ethanol-extracted Powder of Gynostemma pentaphyllum leaf, lactoferrin (milk-purified protein), lemon balm extract-mixed powder, mate hydrothermal extract, and seaweed; complex extracts such as fermented vinegar and pomegranate complex, puer tea extract, black-eyed pea peptide complex, vegetable oil diglyceride, wild mango seed extract, oil containing heavy-chain fatty acid (MCFA), Coleus forskohlii extract, chitooligosaccharide, finger root extract powder, and hibiscus; GABA-containing powder derived from L-glutamic acid, Katsuobushi oligopeptide, natto bacteria-culture powder, black-eyed pea peptide complex, salmon peptide, olive leaf extract, sardine peptide, casein hydrolysate, coenzyme Q10, grape seed enzyme decomposition extract powder, Haitai oligopeptide, etc., which have been recognized for their functionality as ‘blood pressure regulation’; DHA concentrated oil, globin hydrolysate, indigestible maltodextrin, bamboo leaf extract, vegetable oil diglyceride, sardine-refined fish oil, refined squid oil, etc., which have been recognized for their functionality of ‘improving a neutral fat in the blood’; L-arabinose, nopal extract, cinnamon extract powder, guava leaf extract, indigestible maltodextrin, freeze-dried silkworm powder, hemp spirit extract, banaba leaf extract, mulberry leaf extract, etc., which have been recognized for their functionality as ‘blood sugar regulation’; fermentation-generated amino acid complex, Hovenia chinensis fruit extract, Rhodiola extract, etc., which have been recognized for their functionality of ‘alleviating fatigue’; L-theanine, asiaganda extract, milk protein hydrolysate, Gynostemma pentaphyllum leaf extract, etc., which have been recognized for their functionality as ‘anti-stress’.

As an example, the present invention can provide the following embodiments:

A first embodiment can provide a composition for anti-fatigue comprising a green tea peptide composition as an active ingredient, wherein the green tea peptide composition comprises one or more green tea peptides containing an amino acid sequence selected from the group consisting of SEQ ID NO: 1 to SEQ ID NO: 7.

A second embodiment can provide the composition for anti-fatigue according to the first embodiment, wherein the green tea peptide composition is obtained by fermenting a green tea protein with vegetable lactic acid bacterium.

A third embodiment can provide the composition for anti-fatigue according to any one of the first and second embodiments, wherein the vegetable lactic acid bacterium is Lactiplantibacillus plantarum.

A fourth embodiment can provide the composition for anti-fatigue according to any one of the first to third embodiments, wherein the green tea protein is obtained from a residue of a primary extract obtained by extracting a green tea with anhydrous or hydrous C₁-C₆ lower alcohol.

A fifth embodiment can provide the composition for anti-fatigue according to any one of the first to fourth embodiments, wherein a concentration of alcohol in the hydrous C₁-C₆ lower alcohol is 20 to 80% (v/v).

A sixth embodiment can provide the composition for anti-fatigue according to any one of the first to fifth embodiments, wherein the hydrous C₁-C₆ lower alcohol is an ethanol aqueous solution of 20 to 80% (v/v).

A seventh embodiment can provide the composition for anti-fatigue according to any one of the first to sixth embodiments, wherein the green tea protein is obtained from a residue of a secondary extract obtained by extracting the residue of the primary extract with thermal water.

An eighth embodiment can provide the composition for anti-fatigue according to any one of the first to seventh embodiments, wherein the green tea peptide composition inhibits production of a fatigue substance in a muscle cell.

A ninth embodiment can provide the composition for anti-fatigue according to any one of the first to eighth embodiments, wherein the fatigue substance is a lactic acid.

A tenth embodiment can provide the composition for anti-fatigue according to any one of the first to ninth embodiments, wherein the green tea peptide composition exhibits one or more of the following characteristics:
i) characteristic of suppressing expression of any one or more of LDHA and PDK; and
ii) characteristic of increasing expression of PDH.

An eleventh embodiment can provide the composition for anti-fatigue according to any one of the first to tenth embodiments, wherein the green tea peptide composition promotes fat oxidation in the muscle cell.

The twelfth embodiment can provide the composition for anti-fatigue according to any one of the first to eleventh embodiments, wherein the green tea peptide composition increases expression of any one or more of ACO, CPT, mCAD, and PPARα.

A thirteenth embodiment can provide the composition for anti-fatigue according to any one of the first to twelfth embodiments, wherein the green tea peptide composition promotes mitochondrial biogenesis in the muscle cell.

A fourteenth embodiment can provide the composition for anti-fatigue according to any one of the first to thirteenth embodiments, wherein the green tea peptide composition increases expression of any one or more of TFAM, NDUFA9, COX4, and ATP5a.

A fifteenth embodiment can provide the composition for anti-fatigue according to any one of the first to fourteenth embodiments, wherein the green tea peptide composition promotes ATP biosynthesis in the muscle cell.

A sixteenth embodiment can provide the composition for anti-fatigue according to any one of the first to fifteenth embodiments, wherein the green tea peptide composition is contained in an amount of 1 to 50 wt % based on the total weight of the composition for anti-fatigue.

A seventeenth embodiment can provide the composition for anti-fatigue according to any one of the first to sixteenth embodiments, wherein the green tea peptide composition is administered in an amount of 1 to 400 mg/kg/day.

An eighteenth embodiment can provide the composition for anti-fatigue according to any one of the first to seventeenth embodiments, wherein the composition is a pharmaceutical composition or a health functional food composition.

Hereinafter, the contents of the present invention will be described in more detail through Examples and Experimental Examples. However, these Examples and Experimental Examples are provided only to understand the present invention, and the scope of the present invention is not limited to those Examples and Experimental Examples. Any modification, substitution, and insertion commonly known in the art can be carried out, and these are also included in the scope of the present invention.

### [Example 1] Preparation of Green Tea Peptide (GTP) Composition

50 kg of a green tea (Camelia sinensis, Osulloc Farm, an agricultural corporation) was put into a 1-ton extraction tank, and 50% (v/v) ethanol was added to the extraction tank 15 times of the green tea. Thereafter, catechins were removed by extraction (first extraction) and filtration at 70ºC for 2 hours to obtain a residue of a first extract of the green tea. A purified water was added to the residual solid of the obtained first extract of the green tea 15 times of the residual solid, and a water-soluble polysaccharide was removed by extraction (second extraction) and filtration at 90ºC for 3 hours to obtain a residue of a second extract of the green tea. A 2% (w/w) NaOH (98%, Youngjin Co., Ltd.) aqueous solution was added at to the residual solids of the obtained second extract of the green tea 10 times of the residual solid, and extraction (alkali extraction) and filtration were carried out at 70° C. for 3 hours to obtain a filtrate. The obtained filtrate was cooled to a room temperature, and 35% (w/w) hydrochloric acid (Daejeong Chemical) was added to bring a pH to 3.5 to 4.5 or less. After removing a supernatant and washing the precipitate with the purified water 3 to 7 times, the precipitate was spray-dried using an Ohkawara OC-16 spray dryer (inlet 220° C., outlet 90° C.) to obtain a green tea protein with a crude protein content of 50% (w/w) or more. Lactiplantibacillus plantarum APsulloc 331261 medium (containing purified water, vitamin solution, amino acid solution, and mineral solution) containing 1% (w/w) of the green tea protein was placed in an anaerobic fermenter, and culture was performed at pH 6.8 and 37° C. for 48 hours, and then, the culture medium was centrifuged at 4° C. and 10,000 g for 20 minutes (Labogene 1580R (Serial No. KLG4226180220023)) to obtain a supernatant. The obtained supernatant was concentrated in a dry oven at 70° C., and the concentrate was centrifuged at 4° C. and 100,000 g for 1 hour (Hitachi centrifuge CS150NX) to obtain a supernatant. The obtained supernatant was filtered through membrane filtration (pore size 0.22 μm) to obtain a filtrate, and the obtained filtrate was freeze-dried to produce a green tea peptide composition (Green Tea Peptide; GTP) (green tea peptide content 14% (w/w)).

Meanwhile, a sequence of the green tea peptide contained in the green tea peptide composition was analyzed through the following steps:
1) Centrifuging Lactiplantibacillus plantarum APsulloc 331261 medium containing the green tea protein to separate a supernatant, followed by obtaining only the peptide fractionated in the low-molecular-weight peptide section through membrane filtration and size exclusion chromatography;
2) Freeze-drying and de-salting the peptide obtained in the step 1), and then dissolving the peptide in 0.1% (w/w) formic acid and analyzing it by LC-MS/MS, wherein the LC-MS/MS analysis was performed on 3 μg of a sample (based on protein quantification), and the used equipment and analysis condition were as follows;

TABLE 1

| LC equipment | UltiMate 3000 RSLC nano system |
|---|---|
| MS equipment | Q-Exactive Orbitrap HF-X mass spectrometer |
| | (Thermo Fisher Scientific) |
| Column | (trap) Internal diameter: 75 μm × 2 cm, |
| | packed with Acclaim |
| | PepMap 100 C18, 3 μm |
| | (analytical) Internal diameter: |
| | 75 μm × 50 cm, packed with PepMap |
| | RSLC C18, 2 μm |
| Mobile phase | (solvent A) 0.1% (w/w) formic acid (FA) in water |
| | (solvent B) 0.1% (w/w) FA in acetonitrile |
| Mode | Positive ion mode |
| Collision energy | 27% |

3) Based on a spectrum file obtained through the LC-MS/MS analysis in the step 2), searching peptide sequences using the proteome sequence database of the green tea (Camelia sinensis [UniProt Proteome ID: UP000327468]) and the lactic acid bacterium (L. plantarum DSM 20174 [NCBI accession: GCA_014131735.1], L. plantarum APsulloc 331261), wherein the used analysis condition was as follows;

TABLE 2

| | |
|---|---|
| Search engine | MS-GF+ |
| Modifications | (fixed) None |
| | (variable) oxidation (+15.99) of |
| | methionine & acetylation (+42.01) of |
| | the peptide N-terminal |
| m/z tolerance | Precursor: ±10 ppm |
| | Fragment: ±20 ppm |
| False discovery | 1% |
| rate cut-off | |

A sequence of the green tea peptide according to an embodiment of the present invention which was analyzed through the above steps was as follows:

TABLE 3

| | |
|---|---|
| AYKRRKGKFA | SEQ ID NO: 1 |
| FFFFFFFFFFFFFFFYL | SEQ ID NO: 2 |
| ISKIWNSEVPETEVKNEAESP | SEQ ID NO: 3 |
| PFFCEKMMETN | SEQ ID NO: 4 |
| RFLHERMAYYH | SEQ ID NO: 5 |
| RNLNRLQRLLSMKQEYSPRNHLGSRWREY | SEQ ID NO: 6 |
| TTSSRKKEKPRRFWNNHEEVFLITTK | SEQ ID NO: 7 |

### [Experimental Example 1] Confirmation of Efficacy of Inhibiting Production of Fatigue Substance (Lactic Acid) in Muscle Cell by Treatment with Different Concentrations of Green Tea Peptide (GTP) Composition

In general, a cell produces the energy (ATP) needed for survival from mitochondria. After decomposing macronutrients into acetyl-CoA through various assimilation processes, the ATP is produced through the Krebs cycle and the mitochondrial electron transport system in a complex but efficient manner. When the cell is at a resting state, synthesis of the ATP through this manner does not have much effect, but when consumption of the ATP in the cell exceeds production of the ATP due to vigorous exercise, a small amount of the ATP is quickly produced through metabolism of a lactic acid from a pyruvic acid without an efficient but slow oxidation reaction. Through this process, the ATP required for survival of the cell is replenished inefficiently but rapidly, and in return, the lactic acid is accumulated in the cell. The accumulated lactic acid is generally recognized as a fatigue substance because it causes fatigue, muscle pain, etc., and is actually introduced as a fatigue marker and a cell damage marker even in the functional material development guideline established by the Ministry of Food and Drug Safety. In other words, a situation in which accumulation of the lactic acid in the cell is suppressed means that the ATP can be synthesized efficiently without damage in the cell.

The lactic acid is produced from the pyruvic acid by lactate dehydrogenase (LDH). In order to confirm whether the green tea peptide composition (GTP) according to an embodiment of the present invention can suppress production of the lactic acid, the fatigue substance, CoCl₂ was used to make the cell recognize that it is in an oxygen-deficient state and convert to the process of producing the ATP (and the lactic acid) through the LDH without going through an oxidative phosphorylation process.

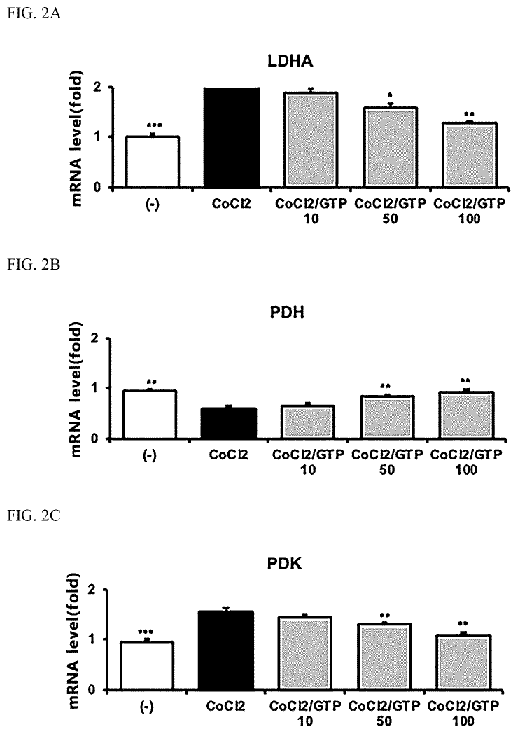

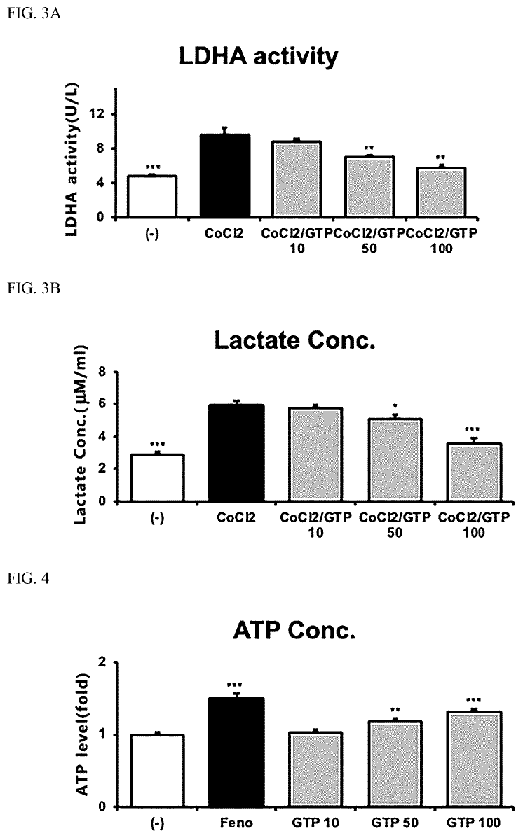

First, the C2C12 muscle cell line (ATCC) was cultured to 100% density with DMEM (Sigma Aldrich)+10% bovine calf serum (Gibco) medium, and then was induced to differentiate into a muscle cell through serum starvation with DMEM+2% horse serum (Gibco) medium. Differentiation into the muscle cell takes about a week, and an end point of the differentiation was set when a multinucleated fiber was produced by differentiating 60% or more of a myoblast into the muscle cell. The differentiated muscle cell were pretreated with the green tea peptide composition (GTP) according to Example 1 at a concentration of 10, 50, and 100 μg/ml for 24 hours, and then treated with CoCl₂ (Sigma Aldrich) of 100 μM for additional 24 hours. To confirm expression of the LDH, etc., RNA extraction and cDNA synthesis were completed using TaKaRa MiniBEST Universal RNA Extraction Kit (Takara Bio) and RevertAid 1^{st}-strand cDNA Synthesis Kit (Thermo Fisher Scientific) sequentially. The synthesized cDNA was used to observe expression of the LDH mRNA with a CFX96 thermocycler (Bio-Rad). The results were shown in FIGS. 2A to 2C, FIGS. 3A and 3B.

As shown in FIGS. 2A to 2C, upon treating with CoCl₂, Expression of LDHA, which produces the lactic acid, was increased, and contrary to this, expression of PDH (pyruvate dehydrogenase), which converts the pyruvic acid into acetyl-CoA to induce production of the ATP through an electron transport chain reaction in the mitochondria, was decreased. In addition, expression of PDK (pyruvate dehydrogenase kinase), which inhibits activity of the PDH, was increased, and this can be seen that CoCl₂ treatment causes the cell to produce the ATP through fermentation of the lactic acid instead of the oxidative phosphorylation process, which leads to accumulation of the lactic acid in the cell. Under this situation, it could be confirmed that in the group pretreated with the green tea peptide composition (GTP) according to Example 1, the effects such as increase in expression of the LDHA due to the CoCl₂ treatment, increase in the PDK, and decrease in the PDH thereby were significantly alleviated.

Further, as shown in FIG. 3A, not only gene expression but also activity of the lactic acid-producing enzyme (Lactate Dehydrogenase Activity Assay Kit, Sigma Aldrich) was also confirmed to be reduced by treatment with the green tea peptide composition (GTP) according to Example 1. Due to such reduction, as shown in FIG. 3B, it could be also confirmed that the accumulation of the lactic acid in the muscle cell (L-Lactate Assay Kit, abcam) was also greatly suppressed. From this, it can be seen that the green tea peptide composition (GTP) according to an embodiment of the present invention would exhibit efficacy of the anti-fatigue reducing accumulation of the lactic acid by suppressing expression and activity of the LDH.

### [Experimental Example 2] Confirmation of Increase in Mitochondria and ATP in Muscle Cell by Treatment with Different Concentrations of Green Tea Peptide (GTP) Composition

From the results of Experimental Example 1, it was confirmed that the green tea peptide composition according to an aspect of the present invention inhibits accumulation of lactic acid in the cell by inhibiting the LDH, which means that the green tea peptide produces the ATP in the method that does not use fermentation of the lactic acid. According to this, in order to confirm whether the green tea peptide composition according to an aspect of the present invention actually promotes production of the ATP in the muscle cell, the green tea peptide composition (GTP) according to Example 1 was added to the differentiated muscle cell in Experimental Example 1 at different concentrations (10, 50, 100 μg/ml) and an amount of the ATP in the cell was measured using a ATP Determination Kit (Invitrogen). The results were shown in FIG. 4. As shown in FIG. 4, it could be found that if the muscle cell was actually treated with the green tea peptide composition, a concentration of the ATP in the cell increased in a concentration-dependent manner.

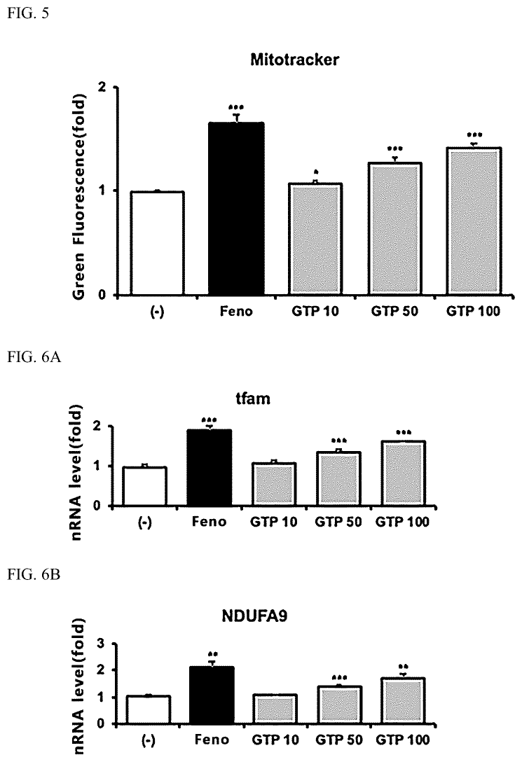

According to this, in order to confirm how the green tea peptide composition increases the ATP, a changes in mitochondria, the ATP synthesis factory within the cell, were observed. The differentiated muscle cells in Experimental Example 1 were treated with the green tea peptide composition according to Example 1 at various concentrations (10, 50, 100 μg/ml), and then, mitochondria in the cell were fluorescently stained using MitoTracker™ Green FM dye (Invitrogen) followed by being quantified using Tecan Infinite M200 Multiplate Reader (Tecan Trading AG; excitation 490 nm, emission 516 nm). The results were shown in FIG. 5.

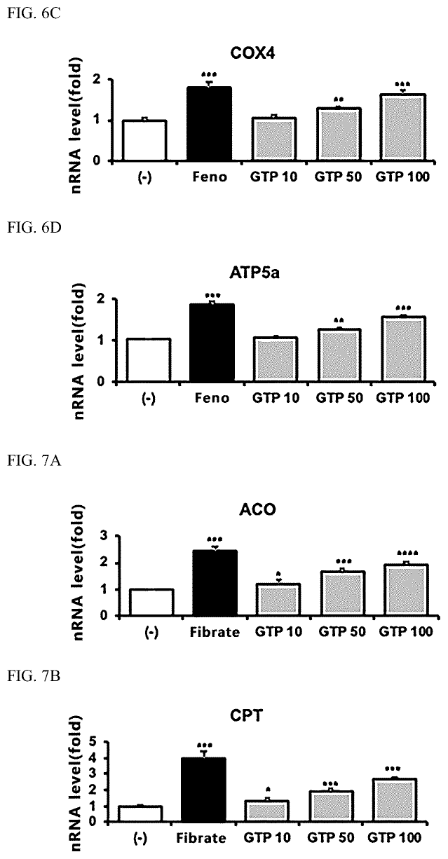

As shown in FIG. 5, when treated with the green tea peptide composition (GTP) according to an aspect of the present invention, it could be confirmed that the number of mitochondria in the muscle cell was significantly increased. In addition, as shown in FIGS. 6A to 6D, it could be observed that expression of mitochondrial and electron transport system genes (tfam, NDUFA9, COX4, ATP5a) in the muscle cell was also increased by treatment with the green tea peptide composition (GTP) according to an aspect of the present invention.

From this, it can be seen that the green tea peptide composition according to an aspect of the present invention would promote synthesis of the ATP by increasing mitochondria in the muscle cell.

### [Experimental Example 3] Confirmation of Efficacy of Green Tea Peptide (GTP) Composition on Promoting Fat Oxidation in Muscle Cell

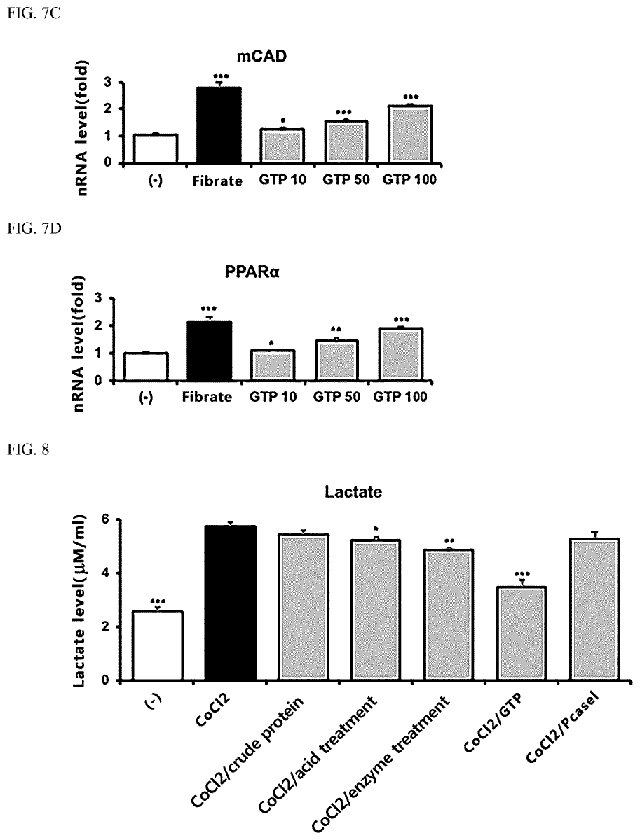

If fat oxidation is promoted and mitochondrial electron transport system is activated, a cell can produce ATP most efficiently. Through Experimental Example 2, it was confirmed that the green tea peptide composition according to an aspect of the present invention increased mitochondria in the muscle cell. Furthermore, in order to additionally investigate whether the green tea peptide composition can also activate fat oxidation, the green tea peptide composition of Example 1 was added to the differentiated muscle cell in Experimental Example 1 at various concentrations (10, 50, 100 μg/ml) in the same manner as in Experimental Example 2, and expression of fatty acid oxidation-related genes (ACO, CPT, mCAD, PPARα) was confirmed in the same method as in Experimental Example 1. The results were shown in FIGS. 7A to 7D.

As shown in FIGS. 7A to 7D, when treated with the green tea peptide composition according to an aspect of the present invention, it could be confirmed that expression of the fatty acid oxidation-related genes in the muscle cell significantly increased in a concentration-dependent manner. Combining this with the results of Experimental Example 2, it can be seen that the green tea peptide composition according to an aspect of the present invention promotes fat oxidation within the muscle cell and increases mitochondria so that metabolites can be better used for synthesis of the ATP, thereby helping the cell produce the large number of the ATP.

### [Experimental Example 4] Confirmation of Efficacy of Green Tea Peptide (GTP) Compositions According to Different Preparation Methods on Inhibiting Accumulation of Lactic Acid in Muscle Cell

An anti-fatigue efficacy of the green tea peptides according to the following four different preparation methods was compared: i) a crude green tea protein obtained during the process of preparing the green tea peptide composition of Example 1; ii) a supernatant (acid-treated fraction of green tea protein) obtained by making a green tea protein obtained during the process of preparing the green tea peptide composition of Example 1 to a concentration of 1:40 (w/v) using purified water, and then adjusting the green tea protein to pH 5 by addition of a hydrochloric acid of 35% (w/w), followed by performing hydrolysis at 37° C. for 6 hours and centrifugation (Labogene 1580R (Serial No. KLG4226180220023)) at 4° C. and 10,000 g; iii) a supernatant (green tea protein enzyme-treated fraction) obtained by making the green tea protein obtained during the process of preparing the green tea peptide composition of Example 1 to a concentration of 1:40 (w/v) using 0.1M sodium phosphate buffer (pH 8), and then adding proteolytic enzyme bromelain at 1% (w/v) compared to the green tea protein, followed by performing hydrolysis at 45° C. and pH 6.2 for 24 hours, heating at 90° C. for 30 minutes and centrifugation (Labogene 1580R (Serial No. KLG4226180220023)) at 4° C. and 10,000 g; and iv) a peptide composition prepared in the same method as that of Example 1, except that Lacticaseibacillus paracasei KCTC 3510 (ATCC strain number: ATCC 25302, Biological Resources Center (KCTC)), an animal lactic acid bacterium, was used instead of Lactiplantibacillus plantarum APsulloc 331261. The specific experimental method was carried out in the same procedure as that of Experimental Example 1, except that the crude green tea protein, the acid-treated fraction of green tea protein, the green tea protein enzyme(bromelain)-treated fraction, Lacticaseibacillus paracasei (L. paracasei)-fermented peptide composition which is the animal lactic acid bacterium, and the green tea peptide composition according to Example 1 were treated at a concentration of 100 μg/ml for 24 hours, respectively. The results were shown in FIG. 8.

From the results of FIG. 8, it could be confirmed that the crude green tea protein and the animal lactic acid bacterium-fermented peptide composition did not show significant efficacy in inhibiting accumulation of the lactic acid, and the acid-treated peptide and the enzyme-treated peptide showed insignificant efficacy, whereas the green tea peptide composition according to an embodiment of the present invention showed the best activity in inhibiting accumulation of the lactic acid among the treated groups.

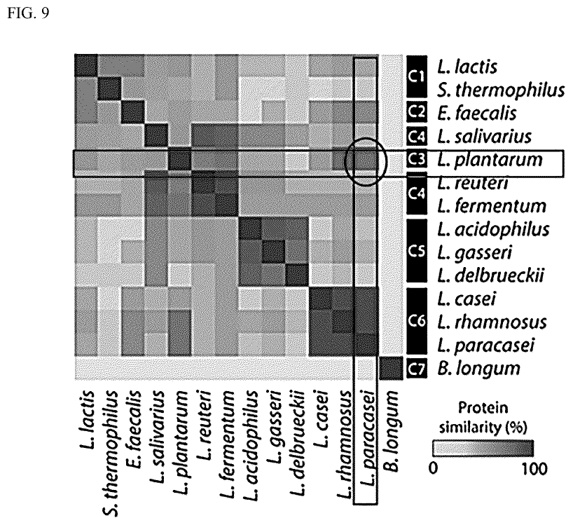

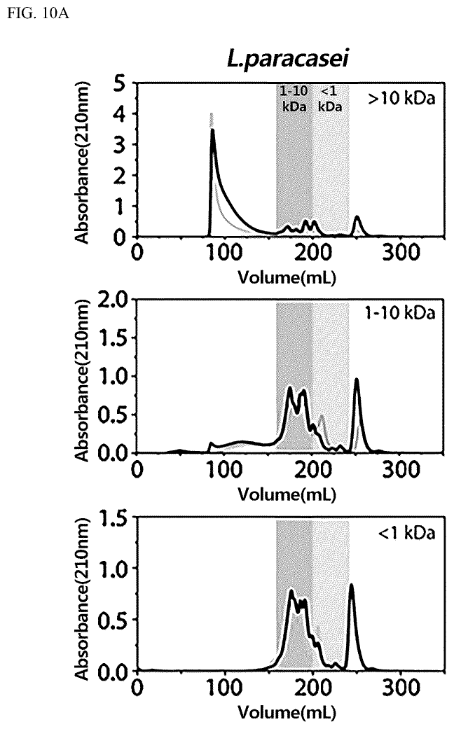

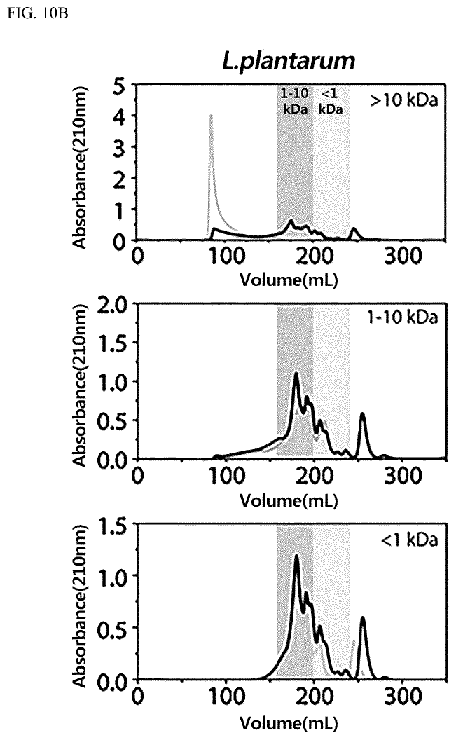

Meanwhile, FIG. 9 shows the results of analyzing the similarity between Lactiplantibacillus plantarum and Lacticaseibacillus paracasei. The similarity analysis was performed by downloading the genome information (genbank) for each strain from NCBI (ncbi.nlm.nih.gov/genbank/), leaving only the annotated conserved protein genetic information and discarding the remaining information, and then calculating as a ratio to the total number counted. In this case, the R program was used to calculate POCP (percentage of conserved proteins) (code source: github.com/hoelzer/pocp.git), and the analysis conditions were as follows:
E-value=1×e⁻⁵
Sequence identity=0.4
Alignment length=0.5.

Among these, if the two genomes showed the similarity of 50% or more, they were classified into the same cluster. Lactiplantibacillus plantarum and Lacticaseibacillus paracasei showed to have a very high similarity of 46.84%. As such, in the case of the green tea peptide composition (PCasei) obtained by fermenting with Lacticaseibacillus paracasei which is the animal lactic acid bacterium very similar to Lactiplantibacillus plantarum used in Example 1, it could be confirmed that the green tea peptide composition did not exhibit efficacy of inhibiting accumulation of the lactic acid, that is, anti-fatigue efficacy. From this, it can be seen that the anti-fatigue efficacy of the green tea peptide composition according to an embodiment of the present invention is obtained through fermentation using Lactiplantibacillus plantarum, a vegetable lactic acid bacterium.

### [Experimental Example 5] Confirmation of Anti-Fatigue Efficacy According to Molecular Weight of Green Tea Peptide (GTP)

As a result of sequence analysis of the peptide isolated and purified from the green tea peptide composition prepared by Example 1, the peptide was identified to have the amino acid sequences of SEQ ID NOS: 1 to 7.

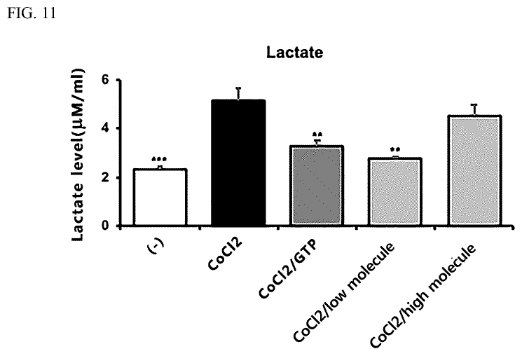

FIGS. 10A and 10B show the results of molecular weight analysis of a green tea protein decomposition products by Lacticaseibacillus paracasei and Lactiplantibacillus plantarum, respectively. Specifically, the molecular weight analysis was performed by a size exclusion chromatography using FPLC (Faste Protein Liquid Chromatography), and specific conditions for the FPLC equipment and a buffer are as follows:

TABLE 4

| | |
|---|---|
| Equipment | BioLogic Duoflow |
| | Chromatography System |
| Column | Superdex 30 pg (200 ml) |
| Buffer | 20 mM Tris-HCl, pH 7.0 |
| Flow rate | 1.5 ml/min |
| Pressure | 7-9 psi |

| | | |
|---|---|---|
| Amount of sample | High molecular weight | 100 mg |
| (concentration) | fraction (>10 kDa) | (50 mg/ml) |
| | Low molecular weight | 100 mg |
| | fraction (≤10 kDa) | (50 mg/ml) |
| | Green tea peptide | 100 mg |
| | composition(GTP) | (50 mg/ml) |

As shown in FIGS. 10A and 10B, it can be confirmed that most of the green tea peptide compositions obtained from fermentation by Lactiplantibacillus plantarum contains the low molecular weight peptides of 10 kDa or less, unlike the green tea peptides (PCasei) fermented by Lacticaseibacillus paracasei.

According to this, the green tea peptide composition prepared by Example 1 was divided into a low molecular weight fraction and a high molecular weight fraction based on the molecular weight of 10 kDa using a dialysis kit (Sigma Aldrich), and each sample was treated at a concentration shown in Table 4 to compare efficacy of inhibiting accumulation of the lactic acid in the muscle cell for each sample in the same manner as in Experimental Examples 1 and 4. The results were shown in FIG. 11.

As shown in FIG. 11, it could be confirmed that the low molecular weight fraction containing a large amount of the novel peptide produced from fermentation by Lactiplantibacillus plantarum showed excellent efficacy of inhibiting accumulation of the lactic acid, whereas the high molecular weight fraction failing to contain the novel peptide did not exhibited efficacy of inhibiting accumulation of the lactic acid. From this, it can be seen that the anti-fatigue efficacy of the green tea peptide composition according to an embodiment of the present invention is caused due to the new peptide decomposed by Lactiplantibacillus plantarum, the fermented lactic acid bacterium.

### [Accession Number]

Name of depository authority: Korea Culture Center of Microorganisms (overseas)

Accession Number: KCCM11179P

Accession Date: 20110328

What is claimed is:

1. A method for preventing, improving, alleviating, recovering, or eliminating fatigue, the method comprising administering to a subject in need an effective amount of a green tea peptide composition comprising one or more green tea peptides containing an amino acid sequence selected from the group consisting of SEQ ID NO: 1 to SEQ ID NO: 7.

2. The method according to claim 1, wherein the green tea peptide composition is obtained by fermenting a green tea protein with vegetable lactic acid bacterium.

3. The method according to claim 2, wherein the vegetable lactic acid bacterium is Lactiplantibacillus plantarum.

4. The method according to claim 2, wherein the green tea protein is obtained from a residue of a primary extract obtained by extracting a green tea with anhydrous or hydrous C1-C6 lower alcohol.

5. The method according to claim 4, wherein a concentration of alcohol in the hydrous C1-C6 lower alcohol is 20 to 80% (v/v).

6. The method according to claim 5, wherein the hydrous C₁-C₆ lower alcohol is an ethanol aqueous solution of 20 to 80% (v/v).

7. The method according to claim 4, wherein the green tea protein is obtained from a residue of a secondary extract obtained by extracting the residue of the primary extract with thermal water.

8. The method according to claim 1, wherein the green tea peptide composition inhibits production of a fatigue substance in a muscle cell.

9. The method according to claim 8, wherein the fatigue substance is a lactic acid.

10. The method according to claim 8, wherein the green tea peptide composition exhibits one or more of the following characteristics:
i) characteristic of inhibiting expression of any one or more of LDHA and PDK; and
ii) characteristic of increasing expression of PDH.

11. The method according to claim 1, wherein the green tea peptide composition promotes fat oxidation in a muscle cell.

12. The method according to claim 11, wherein the green tea peptide composition increases expression of any one or more of ACO, CPT, mCAD, and PPARα.

13. The method according to claim 1, wherein the green tea peptide composition promotes mitochondrial biosynthesis in a muscle cell.

14. The method according to claim 13, wherein the green tea peptide composition increases expression of any one or more of TFAM, NDUFA9, COX4, and ATP5a.

15. The method according to claim 13, wherein the green tea peptide composition promotes ATP biosynthesis in a muscle cell.

16. The method according to claim 1, wherein the green tea peptide composition is formulated as a composition for anti-fatigue, and the green tea peptide composition is contained in an amount of 1 to 50 wt % based on the total weight of the composition for anti-fatigue.

17. The method according to claim 1, wherein the green tea peptide composition is administered in an amount of 1 to 400 mg/kg/day.

18. The method according to claim 1, wherein the green tea peptide composition is formulated as a composition for anti-fatigue, and the composition for anti-fatigue is a pharmaceutical composition or a health functional food composition.


SEQUENCE LISTING (7 sequences)

SEQ ID NO: 1 - protein, 10 aa, unidentified
AYKRRKGKFA

SEQ ID NO: 2 - protein, 17 aa, unidentified
FFFFFFFFFFFFFFFYL

SEQ ID NO: 3 - protein, 21 aa, unidentified
ISKIWNSEVPETEVKNEAESP

SEQ ID NO: 4 - protein, 11 aa, unidentified
PFFCEKMMETN

SEQ ID NO: 5 - protein, 11 aa, unidentified
RFLHERMAYYH

SEQ ID NO: 6 - protein, 29 aa, unidentified
RNLNRLQRLLSMKQEYSPRNHLGSRWREY

SEQ ID NO: 7 - protein, 26 aa, unidentified
TTSSRKKEKPRRFWNNHEEVFLITTK